(19) United States
(12) Plant Patent Application Publication
(10) Pub. No.: US 2015/0089703 P1
(43) Pub. Date: Mar. 26, 2015

(54) Petunia plant named 'Sunpetu 1072'

(50) ;

(71) Applicant: SUNTORY FLOWERS LIMITED, Tokyo (JP)

(72) Inventor: Masahiro Yamada, Kanagawa (JP)

(21) Appl. No.: 14/121,576

(22) Filed: Sep. 19, 2014

Related U.S. Application Data
(60) Provisional application No. 61/960,548, filed on Sep. 20, 2013.

(51) Int. Cl. A01H 5/00

(52) U.S. Cl. PLT/356.14

(57) ABSTRACT

Disclosed herein is a new and distinct variety of Petunia plant having vigorous and trailing habit, abundant flowering, the whole bush remaining in bloom for a considerable period of time, and double, small sized and light greenish yellow colored flowers.

### BOTANICAL DESIGNATION

Petunia×hybrida

### CULTIVAR DENOMINATION

‘Sunpetu 1072’

### CROSS REFERENCE TO RELATED APPLICATIONS

This application is related to and claims priority under 35 U.S.C. 119 to U.S. provisional plant patent application 61/960,548, filed Sep. 20, 2013, entitled ‘Sunpetu 1072.’

### BACKGROUND OF THE INVENTION

The present invention relates to a new and distinct variety of Petunia plant, which is hereinafter referred to as ‘Sunpetu 1072.’

A Petunia is a very popular plant that is used in flower bedding and potting in the summer season.

This purpose of this invention is to obtain a new Petunia cultivar with vigorous and trailing habit, abundant flowering, the whole bush remaining in bloom for a considerable period of time, and double, small sized and light greenish yellow colored flowers.

The new Petunia plant originated from cross-pollination of the female parent ‘MP22-176-1’ and the male parent ‘MP22-138-2’. The female parent ‘MP22-176-1’ (unpatented) used in the crossing of ‘Sunpetu 1072’ is a strain of the applicant's breeding lines (i.e., proprietary Petunia selection), having yellow petals, and the male parent ‘MP22-138-2’ (unpatented) used in the crossing of ‘Sunpetu 1072’ is a strain of the applicant's breeding lines (i.e., proprietary Petunia selection), having white petals. The cross-pollination was conducted in Sep. 10, 2010 at Yame-gun, Fukuoka, Japan.

In March 2011, the seedlings obtained by the crossing were planted in a field. In May 2011, some seedlings were selected in view of growth habit, flower size and color thereof. Shortly thereafter stem tip culturing was carried out, and then the propagation was started.

In November 2012, the cultivation of the seedlings was repeated. The botanical characteristics of that plant were then examined, using similar variety ‘Daiichi-MP21-140-3’ for comparison. As a result, it was concluded that this Petunia plant is distinguishable from any other variety, whose existence is known, and is uniform and stable in its characteristics.

The new variety of Petunia plant was then named ‘Sunpetu 1072’.

### SUMMARY OF THE INVENTION

This new variety is unlike any Petunia commercially available as evidenced by the following unique combinations of characteristics.

1. Vigorous and trailing habit.

2. Abundant flowering, the entire bush remaining in bloom for a considerable period of time.

3. Double, small sized and light greenish yellow colored flowers.

The new variety ‘Sunpetu 1072’ differs from the similar variety ‘Daiichi-MP21-140-3’, which was applied for Japanese plant variety protection (the application number: 26282; the application date: Sep. 8, 2011; the registration number: 22219; the registration date: Jan. 28, 2013 ), in the following points.

1. The leaf shape of ‘Sunpetu 1072’ is ovate. That of ‘Daiichi-MP21-140-3’ is elliptical.

2. The flower diameter of ‘Sunpetu 1072’ is somewhat small (about 5.5 cm). That of ‘Daiichi-MP21-140-3’ is small.

3. The main color of surface of corolla lobe of ‘Sunpetu 1072’ is light greenish yellow (RHS 1B). That of ‘Daiichi-MP21-140-3’ is YELLOW-GREEN (RHS 150).

This new variety of Petunia plant ‘Sunpetu 1072’ was asexially reproduced by the use of cuttings, and homogeneity and stability thereof were confirmed. The instant plant retains its distinctive characteristics and produces true to type in successive generations.

### BRIEF DESCRIPTION OF THE PHOTOGRAPHS

The depicted plants had been reproduced by the use of cuttings and were photographed during October 2013 while growing outdoors in wall pots with about 24 cm of container size at an age of approximately 5 months.

FIG. 1 is a photograph of a typical plant of the new variety of Petunia plant ‘Sunpetu 1072’ while growing in a pot.

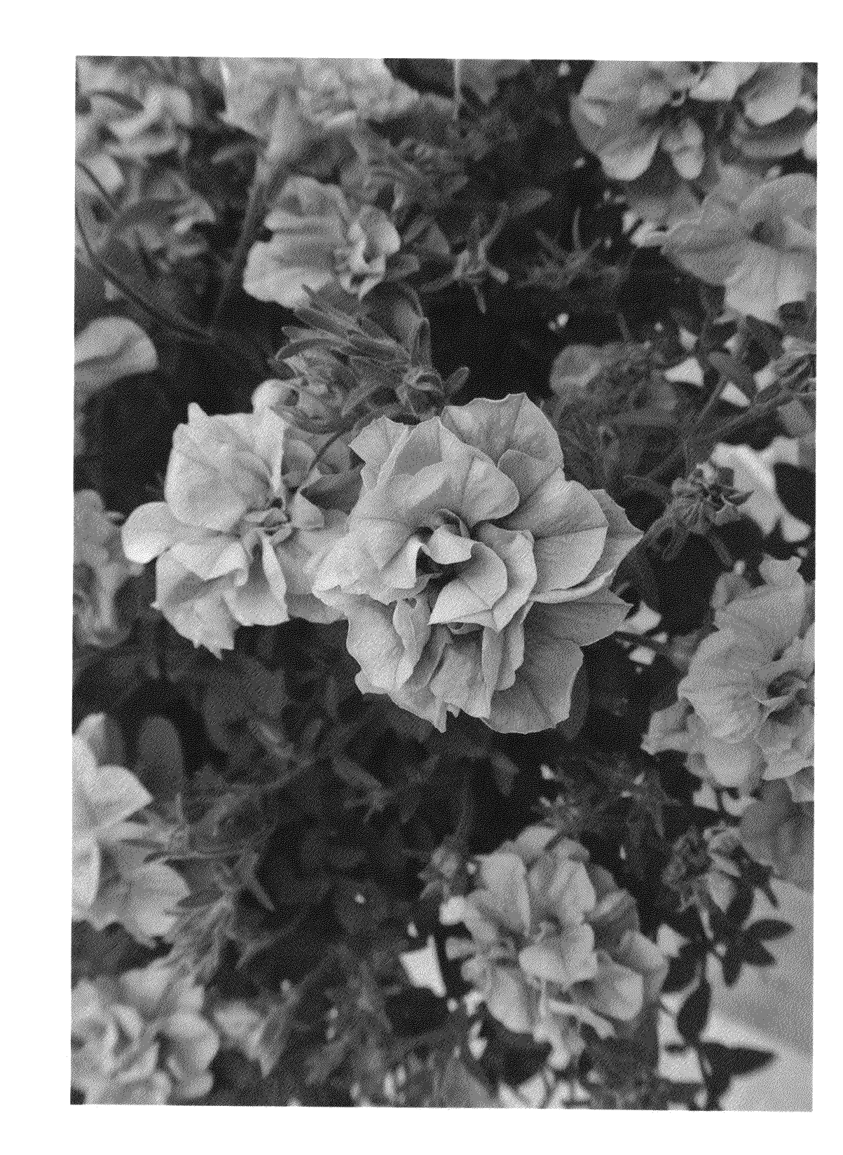

FIG. 2 is a photograph of a close view of flowers of the new variety of Petunia plant ‘Sunpetu 1072’.

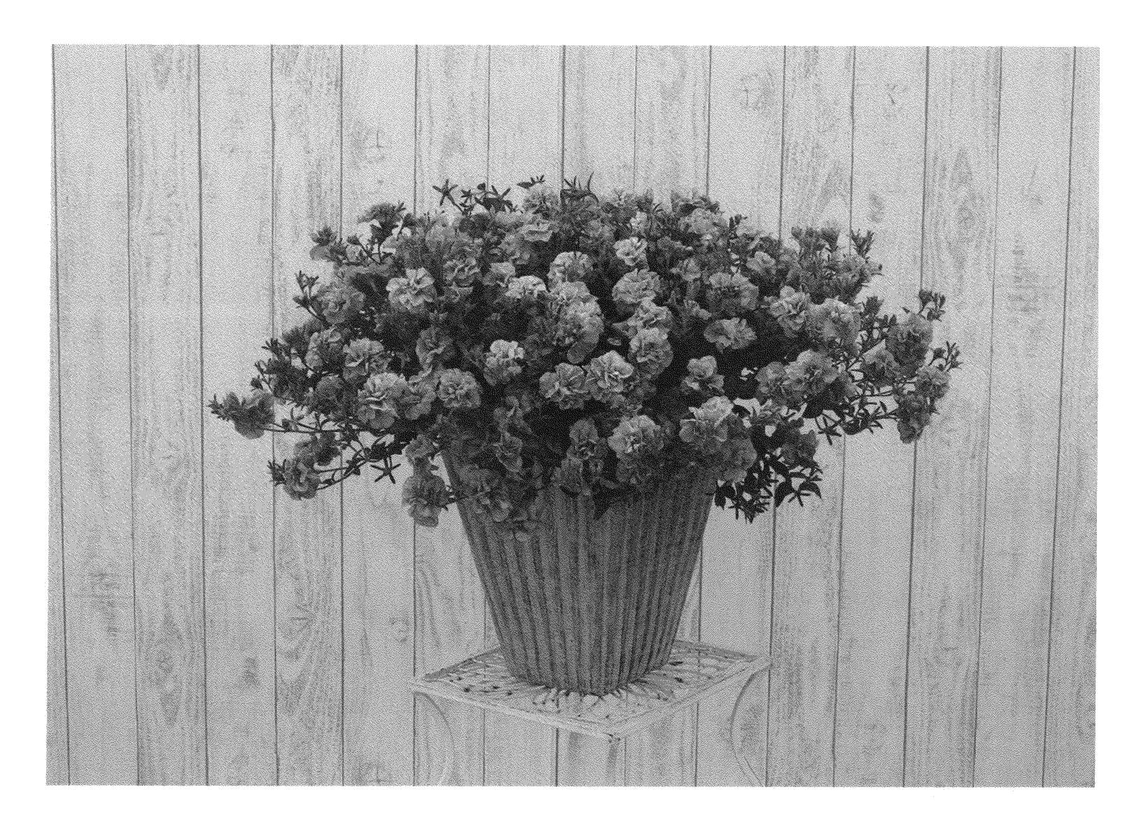

### DETAILED BOTANICAL DESCRIPTION

In October 2013, the cultivation of the seedlings was repeated at Higashiomi, Shiga, Japan. The average day temperature was about 23° C., and the average night temperature was about 13° C. The plants were grown under natural sunlight. The number of days to flowering (response time) was 2-3 weeks. The quality was maintained for about 150 days. The plants had temperature resistance to about −5° C. (the lowest temperature) and about 35° C. (the highest temperature). Further, the plants had good tolerance to pests and pathogens.

For the parentage information: The female parent ‘MP22-176-1’ (unpatented) used in the crossing of ‘Sunpetu 1072’ is a strain of the applicant's breeding lines (i.e., proprietary selection of Petunia×hybrida), having yellow petals; and the male parent ‘MP22-138-2’ (unpatented) used in the crossing of ‘Sunpetu 1072’ is a strain of the applicant's breeding lines (i.e., proprietary selection of Petunia×hybrida), having white petals.

For the propagation information: Approximate soil and/or air temperature was around 15-20° C.; the new cultivar was propagated by cutting; number of days to initiate roots was about 7 days;. number of days to produce a rooted young plant was about 21 days; free-root branching; root color was white; and root texture was fibrous.

The botanical characteristics of the new and distinct variety of Petunia plant named ‘Sunpetu 1072’ at an age of approximately 5 months are shown in the following Table. In the following description, the color-coding is in accordance with the Horticultural Colour Chart of The Royal Horticultural Society, London, England (R.H.S. Colour Chart The 5th edition 2007).

PLANT VARIETY DESCRIPTION

| | APPLICATION VARIETY | |
|---|---|---|
| CHARACTERISTIC | Sunpetu 1072 | |
| ANNUAL, BIENNIAL or PERENNIAL? | Grown as annual | |
| TYPE OF PLANT: i.e., TREE, | Potted plant | |
| SHRUB, SUBSHRUB, VINE, CUT | | |
| FLOWER, POTTED PLANT | | |
| APPROPRIATE CONTAINERS +/or | Ideal for pots, | |
| CROPPING SYSTEM | hanging baskets or | |
| | planters, and is good | |
| | for use as a ground | |
| | cover. | |
| GROWTH HABIT | Semi-trailing | |
| PLANT HEIGHT | About 24.0 | cm |
| PLANT DIAMETER OR AREA OF | About 56.0 | cm |
| SPREAD | | |
| PLANT VIGOR | Vigorous | |
| BRANCHING HABIT | Freely branching | |
| BASAL BRANCHING? | Present | |
| PINCHING REQUIRED? | Required | |
| NUMBER OF LATERAL BRANCHES | Many | |
| LATERAL BRANCH LENGTH | About 20.3 | mm |
| LATERAL BRANCH DIAMETER | About 1.6 | mm |
| INTERNODE LENGTH | About 18.4 | mm |
| STEM ASPECT | Upright to outward | |
| STEM COLOR(and bark color, if | Near 144A | RHS |
| applicable) | | |
| STEM PUBESCENCE? | Pubescent | |
| OTHER PLANT/STEM | — | |
| CHARACTERISTICS | | |
| LEAF ARRANGEMENT | Alternate | |
| COMPOUND OR SIMPLE? | Simple | |
| QUANTITY OF LEAVES PER | Many | |
| LATERAL BRANCH | | |
| LEAF (LEAFLET) SHAPE | Ovate | |
| LEAF (LEAFLET) TIP | Acute | |
| LEAF (LEAFLET) BASE | Obtuse | |
| LEAF LENGTH | About 41.0 | mm |
| LEAF WIDTH | About 28.0 | mm |
| LEAF THICKNESS | About 0.4 | mm |
| LEAF (LEAFLET) TEXTURE | Absent | |
| LEAF PUBESCENCE? WHICH SIDE? | Pubescent, both sides | |
| LEAF (LEAFLET) MARGIN | Entire | |
| VENATION PATTERN | Pinnate | |
| LEAF COLOR, YOUNG, UPPER SIDE | Near 137B | RHS |
| LEAF COLOR, YOUNG, UNDER | Near 137D | RHS |
| SIDE | | |
| LEAF COLOR, MATURE, UPPER | Near 137B | RHS |
| SIDE | | |
| LEAF COLOR, MATURE, UNDER | Near 137D | RHS |
| SIDE | | |
| VENATION COLOR, UPPER SIDE | Near 138B | RHS |
| VENATION COLOR, UNDER SIDE | Near 138C | RHS |
| PETIOLE LENGTH | About 6.6 mm | |
| PETIOLE DIAMETER | About 1.8 mm | |
| PETIOLE TEXTURE | Pubescent | |
| PETIOLE COLOR | Near 138C RHS | |
| STIPULES, TENDRILS, THORNS, | — | |
| SPINES OR PRICKLES? IF SO, | | |
| GIVE COLOR AND SIZE | | |
| OTHER FOLIAGE | — | |
| CHARACTERISTICS | | |
| FLOWER ARRANGEMENT | Borne in upper leaf | |
| | axils | |
| INFLORESCENCE TYPE OR FORM | Flower solitary | |
| (if applicable) | | |
| FLOWER TYPE or FORM | Double | |
| FLOWERING HABIT | Continuously | |
| QUANTITY OF FLOWERS PER | About 1 to 3 | |
| LATERAL STEM | | |
| QUANTITY OF FLOWERS PER | About 60 | |
| PLANT | | |
| NATURAL FLOWERING SEASON | About Spring to late Autumn | |
| TIME TO FLOWER OR RESPONSE | About 2~3weeks | |
| TIME | | |
| FRAGRANCE | Absent | |
| FLOWER BUD LENGTH | About 20.7 mm | |
| FLOWER BUD DIAMETER | About 16.6 mm | |
| FLOWER BUD SHAPE | Obconical | |
| FLOWER BUD COLOR | Near 145B RHS | |
| FLOWER ASPECT; i.e., UPRIGHT, | Upright to outward | |
| OUTWARD, DROOPING, etc. | | |
| FLOWER SHAPE | Salver form | |
| FLOWER DIAMETER | About 55.0 | mm |
| FLOWER DEPTH (HEIGHT) | About 37.0 | mm |
| FLOWER LONGEVITY ON PLANT | About 7~10 days | |
| PERSISTENT OR SELF-CLEANING? | Self-cleaning | |
| PETAL TEXTURE, UPPER SURFACE | Mat | |
| PETAL TEXTURE, LOWER SURFACE | Mat | |
| TUBE TEXTURE, | Pubescent | |
| PETAL ARRANGEMENT | Single whorl | |
| PETAL NUMBER | 5 | |
| PETALS FUSED? | Fused | |
| PETAL SHAPE | — | |
| PETAL MARGIN | Undulated | |
| PETAL TIP | Mucronate | |
| PETAL BASE | Fused | |
| PETAL LENGTH | About 15.7 | mm |
| PETAL WIDTH | About 17.2 | mm |
| PETAL COLOR, WHEN OPENING, | Near 150C with 154A~154C | RHS |
| UPPER SIDE | vein | |
| PETAL COLOR, WHEN OPENING, | Near 145C | RHS |
| LOWER SIDE | | |
| PETAL COLOR, FULLY OPENED, | Near 1B with 154A~154C | RHS |
| UPPER SIDE | vein | |
| PETAL COLOR, FULLY OPENED, | Near 1C | RHS |
| LOWER SIDE | | |
| PETAL COLOR, FADING TO | About - | |
| THROAT DIAMETER | About 14.1 | mm |
| TUBE DIAMETER (PROXIMAL END) | About 4.7 | mm |
| TUBE LENGTH | About 22.0 | mm |
| THROAT COLOR (inside) | Near 149D | RHS |
| TUBE COLOR (outside) | Near 1C | RHS |
| SEPAL ARRANGEMENT | Single whorl | |
| NUMBER OF SEPALS | 5 | |
| SEPAL SHAPE | Narrow elliptic | |
| SEPAL MARGIN | Entire | |
| SEPAL TIP | Obtuse | |
| SEPAL BASE | Fused | |
| SEPAL LENGTH | About 12.1 | mm |
| SEPAL WIDTH | About 3.3 | mm |
| SEPAL COLOR, IMMATURE, UPPER | Near 137B | RHS |
| SIDE | | |
| SEPAL COLOR, IMMATURE, UNDER | Near 137C | RHS |
| SIDE | | |
| SEPAL COLOR, MATURE, UPPER | Near 143A | RHS |
| SIDE | | |
| SEPAL COLOR, MATURE, UNDER | Near 143B | RHS |
| SIDE | | |
| CALYX SHAPE | Tubular, star shape | |
| CALYX LENGTH | About 12.0 | mm |
| CALYX DIAMETER | About 24.9 | mm |
| PEDUNCLE LENGTH | About 22.6 | mm |
| PEDUNCLE DIAMETER | About 1.3 | mm |
| PEDUNCLE ANGLE | Upright to outward | |
| PEDUNCLE TEXTURE | Pubescent | |
| PEDUNCLE COLOR | Near 137D | RHS |
| STAMEN NUMBER | 5 | |
| STAMEN LENGTH | About 15.9 | mm |
| STAMEN LENGTH | About 8.9 | mm |
| ANTHER SHAPE | Ellipsoidal | |
| ANTHER SIZE? | About 2.5 × 1.8 | mm |
| ANTHER COLOR | Near 144A | RHS |
| AMOUNT OF POLLEN | Moderate | |
| POLLEN COLOR | Near 10D | RHS |
| PISTIL NUMBER | Absent | |
| PISTIL LENGTH | About - | mm |
| STIGMA SHAPE | — | |
| STIGMA COLOR | Near - | RHS |
| STYLE COLOR | Near - | RHS |
| OVARY COLOR | Near - | RHS |
| OTHER FLOWER | — | |
| CHARACTERISTICS | | |
| QUANTITY OF SEEDS | Seed production has | |
| | not been observed. | |
| ROOT STRUCTURES such as BULBS, | Fibrous root | |
| CORMS or RHIZOMES? | | |
| LOW TEMPERATURE TOLERANCE | Good | |
| HIGH TEMPERATURE TOLERANCE | Good | |
| DISEASE RESISTANCE AND/OR | Good | |
| SUSCEPTIBILITY | | |
| RESISTANCE OF PESTS AND/OR | Good | |
| SUSCEPTIBILITY | | |

This new variety of Petunia plant having the above botanical characteristics is suitable for flower bedding and potting, particularly in hanging pots or planters.

It is claimed:

1. A new and distinct variety of Petunia plant named ‘Sunpetu 1072’, substantially as herein illustrated and described.
